(19) United States
(12) Patent Application Publication
(10) Pub. No.: US 2002/0081311 A1
(43) Pub. Date: Jun. 27, 2002

(54) Probiatic product

(76) Inventors: Fergus Shanahan, Cork (IE); John Kevin Collins, Cork (IE); Barry Kialy, Cork (IE); Colum Dunne, Cork (IE); Gerald O'Sullivan, Cork (IE); Liam O'Mahony, Cork (IE)

Correspondence Address: JACOBSON, PRICE, HOLMAN & STERN, PROFESSIONAL LIMITED LIABILITY, 400 SEVENTH STREET N.W., WASHINGTON, DC, 20004, US

(21) Appl. No.: 09/853,670

(22) Filed: May 14, 2001

Related U.S. Application Data
(63) Continuation of application No. PCT/IE00/00063, filed on May 12, 2000.

(51) Int. Cl. A61K 39/02; C12N 9/52; A61K 39/09; C12N 1/20

(52) U.S. Cl. 424/190.1; 435/220; 435/252.9

(57) ABSTRACT

An adherence factor comprises a cell wall associated adhesin derived from Lactobacillus or a derivative, fragment, precursor or mutant of the adhesin, the adherence factor mediating adherence to epithelial cells and modulating gene expression to improve gut barrier function and gastrointestinal tract homeostasis.

The Lactobacillus is Lactobacillus salivarius subspecies Salivarius. The adhesin has a molecular weight of 83 kDa.

The invention relates to probiotic material and in particular to probiotic materials derived from Lactobacillus salivarius.

Consumers are becoming increasingly aware of matters which may be necessary for maintenance of their environment, health and nutrition. In response, scientific research has focused upon the roles that diet, stress, and modern medical practices (e.g. antibiotics and radiotherapy) may play in threatening human health. In particular, population dynamics shifting towards older societies are increasing the incidence of illnesses which may be caused by deficient or compromised microflora such as gastrointestinal tract (GIT) infections, constipation, irritable bowel syndrome (IBS), inflammatory bowel disease (IBD)—Crohn's disease and ulcerative colitis, food allergies, antibiotic-induced diarrhoea, cardiovascular disease, and certain cancers (e.g. colorectal cancer).

Probiotics have been defined as live microbial food supplements which beneficially affect the host by improving the intestinal microbial balance, or more broadly, as living micro-organisms, which upon ingestion in certain numbers, exert health effects beyond inherent basic nutrition. Cocktails of various micro-organisms, particularly species of Lactobacillus and Streptococcus, have traditionally been used in fermented dairy products to promote health.

In recent years the commercial manufacture and marketing of functional foods (foods which affect functions of the body in a targeted manner so as to bring about positive affects on physiology and nutrition), particularly probiotic (Acidophilus-Bifidus) yoghurts, has spread from the well-established Japanese niche market place into the lucrative and expanding European Union. While a number of probiotic bacteria of human origin are now being exploited commercially (e.g., L. acidophilus LA-1), many consumers, consumer organisations, and members of the scientific community are sceptical of such products and their publicised probiotic claims. The dairy-food industry is therefore under considerable pressure to scientifically validate these new probiotic food products.

Criteria which have been suggested for the selection of potentially effective probiotic micro-organisms may be summarised as follows: human origin, non-pathogenic behaviour, resistance to technological processes (i.e., viability and activity in delivery vehicles), resistance to gastric acidity and bile toxicity, adhesion to gut epithelial tissue, ability to colonise the GIT, production of antimicrobial substances, ability to modulate immune responses, ability to persist, albeit for short periods, in the gastrointestinal tract and the ability to influence metabolic activities (e.g., cholesterol assimilation, lactase activity, vitamin production (37).

Some Lactobacilli are indigenous to the intestinal tract of man and animals. Such Lactobacilli have traditionally been used in fermented dairy products to promote human health through the influences they may exert on the microbial ecology of the host, lactose intolerance, incidence of diarrhoea, mucosal immune response, levels of blood cholesterol, and cancer (1, 2)

A number of research groups have published reports describing in vitro assays which facilitate the evaluation of microorganisms to epithelial cells of animal (5, 6) and human origin (7, 8, 9, 10).

Other research groups have described assays evaluating bacterial adhesion to intestinal mucus (11) or to synthetic moieties (12).

Many of the reported studies focused upon the evaluation of bacterial adhesion to epithelial cells and have utilised HT-29 and Caco-2 cells, which are human intestinal cell-lines expressing morphological and physiological characteristics of normal human enterocytes (13). These cell-lines have been exploited extensively to elucidate the mechanisms mediating enteropathogen adhesion (14, 15).

In more recent studies, however, HT-29 and Caco-2 cells have been employed in order to select for, and subsequently assess, lactic acid bacteria on the basis of their adherence properties (16, 17, 18, 19, 20, 21, 22).

Further studies involving such probiotic Lactobacilli have demonstrated:

i) that following oral administration the introduced bacteria can be recovered from biopsy specimens of colonic mucosa (23, 24).

ii) competitive exclusion, even by heat-killed bacterial cells, of potential microbial pathogens from human epithelial cells and mucus (25, 26, 27, 28).

In order to determine the mechanisms mediating the interactions that occur between bacterial cells and the surrounding environment and, thereby, the probiotic traits described above, scientists have begun to elucidate the taxonomy, physiology and genetic properties of probiotic bacteria (29, 30, 31). These studies have implicated a number of factors in the attachment of probiotic bacterial cells to epithelial cells. Such factors include:

i) passive entrapment of the bacterial cells by fimbrial cell matrix material (20);

ii) bacterial cell surface-associated lipotechoic acid(33);

iii) proteinaceous extracellular adhesins (6, 8, 17, 18);

iv) bacterial cell surface-associated proteinaceous factors (34, 19).

There is a need to identify the factors involved in the adhesion of probiotic bacterial cells to epithelial cells which will have particular beneficial effects on nutrition, therapy and on health in general.

Statements of Invention

According to the invention there is provided an adherence factor comprising a cell wall associated adhesin derived from a Lactobacillus or a derivative, fragment precursor or mutant of the adhesin, the adherence factor mediating adherence to epithelial cells and modulating epithelial gene expression to improve gut barrier function.

In one embodiment of the invention expression of any one or more of a cadherin, a semaphorin, wnt-13, tenascin or an integrin is upregulated. Most preferably expression of a cadherin is upregulated. Cadherins are the prime mediators of epithelial cell-cell adhesin.

In another embodiment of the invention expression of any one or more of ras-related C3 botulinum toxin substrate 1 (Rac) or TNFα is downregulated.

Preferably the Lactobacillus is isolated from resected and washed human gastrointestinal tract, preferably the Lactobacillus is Lactobacillus salivarius, most preferably Lactobacillus salivarius subspecies Salivarius. The Lactobacillus may be Lactobacillus salivarius subspecies Salivarius UCC118 or a mutant or variant thereof.

Preferably the adherence factor is proteinaceous in nature.

Preferably the factor has a molecular weight of approximately 83 kDa.

Most preferably the factor has at least portion of the N-terminal amino acid sequence listed in SEQ. ID. No. 1.

In one embodiment of the invention the Lactobacillus is in the form of viable cells. Alternatively the Lactobacillus may be in the form of non-viable cells.

The invention further provides a formulation which comprises a factor of the invention.

Preferably the formulation comprises a probiotic material. Alternatively or additionally the formulation comprises a probiotic material.

In one embodiment of the invention the formulation comprises a strain of Streptococcus thermophilus.

In one embodiment of the invention the formulation comprises an ingestable carrier, preferably the ingestable carrier is a pharmaceutically acceptable carrier such as a capsule, tablet or powder, most preferably the ingestable carrier is a food product such as acidified milk, yoghurt, frozen yoghurt, milk powder, milk concentrate, cheese spreads, dressings or beverages.

In one embodiment of the invention the formulation comprises a protein and/or peptide, in particular proteins and/or peptides that are rich in glutamine/glutamate, a lipid, a carbohydrate, a vitamin, mineral and/or trace element.

Preferably the formulation comprises an adjuvant. The formulation may comprise a bacterial component. The formulation may alternatively or additionally comprise a drug entity. The formulation may also comprise a biological compound.

The formulation may be in an orally ingestable form.

The invention further provides a factor or formulation for use in foodstuffs or for use as a medicament.

The product or formulation may be for use in the prophylaxis and/or treatment of undesirable inflammatory activity.

The invention provides use of Lactobacillus bacteria isolated from resected and washed human gastrointestinal tract or its cell wall associated adhesin or derivative, fragment, precursor, mutant or recombinant products thereof for improving gut barrier function and or competitively excluding potential pathogens from binding to and or invading epithelial cells.

The invention also provides use of Lactobacillus bacteria isolated from resected and washed human gastrointestinal tract or its cell wall associated adhesin or derivative, fragment, precursor, mutant or recombinant products thereof for mediating adherence of microorganisms to epithelial cells.

Preferably the Lactobacillus is Lactobacillus salivarius, preferably Lactobacillus salivarius subsp. Salivarius strain, most preferably Lactobacillus salivarius subsp. Salivarius strain UCC118.

The invention further provides Lactobacillus salivarius subsp. Salivarius strain or its adhesin component or recombinant products bearing all or part of the adhesin amino acid sequence SEQ. ID. No. 1 for use in engineering hyper-adhesive variants of microorganisms.

One aspect of the invention provides a vaccine comprising an adherence factor or formulation of the invention.

A further aspect provides use of an adherence factor of the invention for the preparation of a medicament for use in generating an immune response, for engineering hyperadhesive mutants, for the preparation of a medicament or for use in regulating cell cycle and/or invasive behaviour of tumour cells.

The invention further provides a delivery system for delivery of borne factors to intestinal tissue comprising a factor of the invention.

One aspect of the invention provides Lactobacillus salivarius subsp. Salivarius strain UCC118 or its adhesin component or recombinant products bearing all or part of the adhesin amino acid sequence SEQ. ID. No. 1 for use in generating an immune response in inflamed and/or non-inflamed intestinal tissue, for use as a vaccine, for use in the delivery of borne factors to inflamed and/or non-inflamed intestinal tissue and persistence at the sites of adherence to allow slow-release of the borne factors, or for use in foods or medicaments.

The invention also provides a cell wall associated adhesin having a molecular weight of approximately 83 kDa.

The invention further provides a cell wall associated adhesin containing the N-terminal amino acid sequence listed in SEQ. ID. No. 1.

The adherence factor of the invention additionally or alternatively competitively excluding potential pathogens from binding to and or invading epithelial cells, the factor comprising a cell wall associated adhesin or derivative, fragment, precursor or mutant thereof, which mediates adherence of microorganisms to epithelial cells and being derived from Lactobacillus salivarius isolated from resected and washed human gastrointestinal tract.

### BRIEF DESCRIPTION OF THE DRAWINGS

FIG. 1 is a bar chart showing the adherence of individual probiotic Lactobacillus or Bifidobacterium strains when introduced onto either HT-29 or CaCo-2 epithelial cell monolayers;

FIG. 2 is a Scanning Electron Micrograph (SEM) showing the adherence of probiotic Lactobacillus salivarius UCC118 cells to HT-29 epithelial cell monolayer;

FIG. 3 is a bar chart showing the adherence of probiotic Lactobacillus salivarius UCC118 cells to HT-29 epithelial cell monolayer. It is noted that the bacterial cells adhere at a greater level when introduced onto differentiated epithelial cells;

FIG. 4 is a bar chart showing the difference in adherence of probiotic Lactobacillus salivarius UCC118 cells to HT-29 epithelial cell monolayer during log phase and stationary phase of bacterial growth;

FIG. 5 is a bar chart showing the adherence of probiotic Lactobacillus salivarius UCC118 cells to HT-29 epithelial cell monolayer. It is noted that the adherence of the bacterial cells is mediated by the presence of a proteinaceous, cell-associated factor; and that this trait can be negatively influenced by treatment of the bacterial cells with Trypsin;

FIG. 6 is a Gel electrophoresis (PAGE) of Lactobacillus salivarius UCC118 proteinaceous, cell-associated factors. Two protein bands are particularly distinct with approximate molecular weights of 190 kDa and 83 kDa, respectively;

FIG. 7 shows a graph of the purification of a proteinaceous, cell-associated factors from Lactobacillus salivarius UCC118 by DEAE-Sephacel anion exchange chromatography. Analysis of the protein content of the collected FPLC fractions shows obvious peaks at fractions 8, 12, 18, 20, 32 and 38;

FIG. 8 is a bar chart showing the adherence of probiotic Lactobacillus salivarius UCC118 cells to HT-29 epithelial cell monolayer. It is noted that the adherence of the bacterial cells is significantly reduced when FPLC fraction 18 is added prior to the bacterial cells. FPLC fractions 20, 32, 38 and 48 do not significantly affect adherence of Lactobacillus salivarius UCC118 cells;

FIG. 9 is a Gel electrophoresis (PAGE) of a Lactobacillus salivarius UCC118 proteinaceous, cell-associated factor present in FPLC fraction 18. This protein band corresponds with the protein band having an approximate molecular weight of 83 kDa seen in FIG. 6;

FIG. 10 is a bar chart showing the invasion of HT-29 epithelial cell monolayer by a strain of Listeria monocytogenes to be significantly inhibited by the presence of probiotic Lactobacillus salivarius UCC118 cells;

FIG. 11 is a bar chart showing the adherence to HT-29 epithelial cell monolayer by a strain of Enterococcus. The adherence can be significantly inhibited by the presence of probiotic Lactobacillus salivarius UCC118 cells.

FIG. 12 is a bar chart showing the adherence to human intestinal mucosa by probiotic Lactobacillus salivarius UCC118 cells as determined by microbiological analysis of biopsy specimens; and

FIG. 13 is a table showing the number of probiotic Lactobacillus salivarius UCC118 cells adherent to human intestinal mucosa as determined by microbiological analysis of biopsy and faecal specimens. It is noted that UCC118 is capable of persisting within the human gastrointestinal environment for a period of at least 24-26 days.

### DETAILED DESCRIPTION

We have identified an adherence factor, derived from Lactobacillus salivarius isolated from resected and washed human gastrointestinal tissue, which has been found to improve gut barrier function by modulation of epithelial gene expression.

Gut barrier function relates to the ability of the gastrointestinal epithelial monolayer to exclude luminal contents from entering the lamina propria and subsequently interacting with systemic processes. Luminal contents to be excluded include, but are not limited to, bacteria, fungi, yeasts, metabolites and ingested particulate matter. A disturbance of gut barrier function allows invasion of microbes, metabolites, etc. normally contained within the lumen, into the underlying intestinal layers resulting in tissue damage and inflammation. Enhancement of genes controlling epithelial cell-cell binding and cellular integrity would enhance the ability of the gastrointestinal monolayer to resist damage and would promote healing of damaged cells. Thus, interaction between at least an adherence factor derived from Lactobacillus UCC118 and gastrointestinal epithelial cells promotes gut barrier function and is useful in prophylactic and therapeutic settings.

The adherence factor of the invention has been found to result in the up-regulation of epithelial genes such as cadherins, semaphorins, wnt-13, tenascin and integrins thereby improving gut barrier function and gastrointestinal homeostasis. Cadherins are the prime mediators of epithelial cell-cell adhesion. Semaphorins play key roles in the control of cellular interactions, while wnt-13 is a developmental protein affecting development of discrete regions of tissue. Tenascin regulates epithelial differentiation and integrins play multiple roles in cell differentiation and cell-cell interactions.

The adherence factor of the invention has also been found to reduce levels of genes such as the ras-related C3 botulinum toxin substrate 1 (Rac) involved in the invasive behaviour of tumour cells and the TNFα gene which is a proinflammatory cytokines.

Alternatively or additionally the adherence factor of the invention has been found to competitively exclude potential pathogens from binding to and or invading epithelial cells and which mediates in the adherence of microorganisms to epithelial cells.

The product therefore has potential application in a wide range of treatments including improving gut barrier function and gastrointestinal homeostasis and reducing tumour invasiveness and inflammatory responses within the gut.

The product is derived from Lactobacillus salivarius subspecies Salivarius UCC118. A deposit of Lactobacillus salivarius strain UCC 118 was made at the NCIMB on Nov. 27, 1996 and accorded the accession number NCIMB 40829. The strain of Lactobacillus salivarius is described in WO-A-98/35014.

UCC118 isolated from resected and washed human gastrointestinal tract has been found to adhere to epithelial cells in vitro and competitively exclude potential pathogens from binding to and or invading the epithelial cells. UCC118 has been found to adhere to both inflamed and non-inflamed intestinal tissue and remains detectable for a period of at least 12 days post cessation of oral administration. This may have potential application in allowing delivery of product borne factors to inflamed and/or non-inflamed intestinal tissue and persistence at the sites of adherence may allow the slow release of the borne factors. Such product borne factors may include suitable pharmaceutical compounds.

We have isolated a factor which has shown a significant reduction of adhesion of UCC118 when added prior to the introduction of the bacterial strain.

The factor is proteinaceous in nature with a molecular weight of approximately 83 kDa as determined by 10% SDS PAGE. It has an N-terminal amino acid sequence as listed in SEQ ID NO. 1

The adhesin factor described has potential application in a wide range of treatments. In particular, Lactobacillus salivarius subsp. Salivarius strain UCC118 or its adhesin component or recombinant products bearing all or part of the adhesin amino acid sequence may have use in engineering hyper-adhesive mutants, in particular hyper adhesive variants of UCC18 or other microorganisms; generating an immune perception in inflamed and/or non-inflamed intestinal tissue; vaccination; delivery of borne factors to inflamed and/or non-inflamed intestinal tissue and persistence at the sites of adherence which may allow slow-release of the borne factors; regulating cell cycle and invasive behaviour of tumour cells and in foods or medicaments.

The product of the invention may be administered in an orally injestable form in the conventional form of preparation such as capsules, microcapsules, tablets, granules, powder, troches, pills, suppositories, injections, suspensions and syrups. Suitable formulations may be prepared by methods commonly employed using conventional organic and inorganic additives. The amount of active ingredient in the medical composition may be at a level that will exercise the desired therapeutic effect.

In addition a vaccine comprising product of the invention may be prepared using any suitable known method and may include a pharmaceutically acceptable carrier or adjuvant.

The invention will be more clearly understood from the following examples.

### EXAMPLE 1

Isolation of Lactobacillus salivarius Subsp. salivarius Strain UCC118

Lactobacillus salivarius subsp. salivarius strain UCC 118 was isolated from washed specimens of healthy gastrointestinal mucosa removed from the terminal ileum of a normal human (elderly female) gastrointestinal tract during urinary tract reconstructive surgery.

UCC118 was identified as Lactobacillus salivarius based on the results of API™ 50CHL (API™ systems, BioMerieux SA, France) system which tentatively identified the Lactobacillus species by its carbohydrate fermentation profile. Overnight MRS cultures were harvested by centrifugation and resuspended in the suspension medium provided by the manufacturer. API™ strips were inoculated and analysed after 24-48 hours according to the manufacturers instructions. SDS-polyacrylamide gel electrophoresis analysis (SDS-PAGE) of total cell protein further determined the identity of UCC118 as a strain of Lb. salivarius subsp. Salivarius.

### EXAMPLE 2

Assessment of Adhesion by the Lactobacillus Strain to Epithelial Cells in vitro

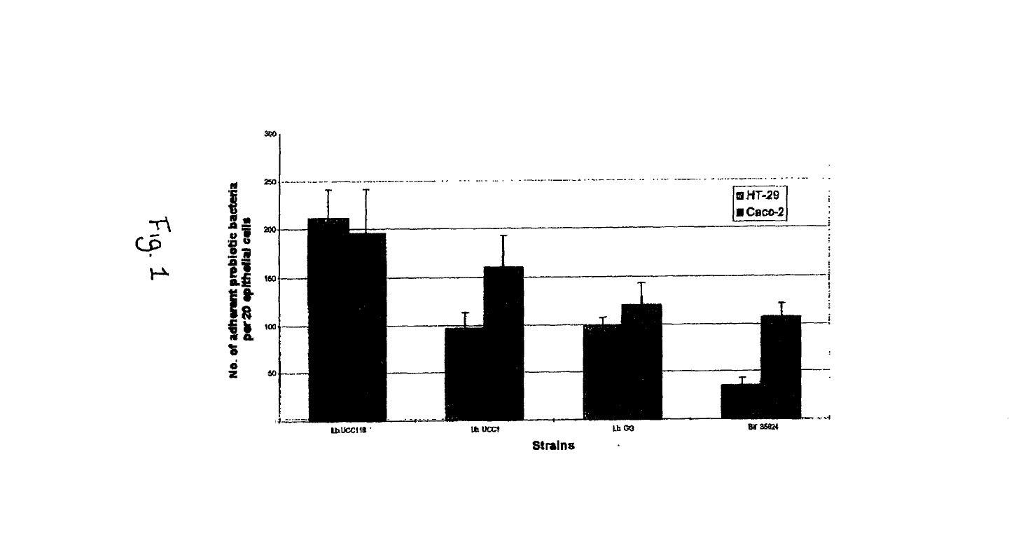

Caco-2 and HT-29 enterocytic cell-lines (14, 35) were cultured as monolayers in DMEM (Dulbeccos modified Eagle's medium: Gibco Ltd., Paisley, Scotland) supplemented with 10% (v/v) foetal calf serum (Gibco Ltd.). Cells were grown in 75 cm² tissue culture flasks (Costar, Cambridge, Mass., USA) at 37° C. in a humidified atmosphere containing 5% CO₂. At 95% confluency the monolayers were passaged by incubating with a 0.25% trypsin solution (Gibco) for 10 min at 37° C. The adhesion of the strains was examined using a modified version of a previously described method (25) (FIG. 1).

Briefly, monolayers of Caco-2 and HT-29 cells were prepared on sterile 22 mm² glass coverslips, which were placed in tissue culture dishes. The cells were seeded at a concentration of 4×10⁴ cells/cm² and fed fresh medium every 2 days for a maximum of 10 days. The Caco-2 and HT-29 monolayers were washed twice with phosphate buffered saline (PBS). Antibiotic free DMEM (2 ml) and 2 ml of bacterial suspension (containing approx. 10⁹ cfu/ml) were added to each dish and cells were incubated for 90 min at 37° C. in a humidified atmosphere containing 5% CO₂. After incubation the monolayers were washed five times with sterile PBS, fixed with methanol for 3 min, Gram stained and examined microscopically under oil immersion. For each glass coverslip monolayer the number of adherent bacteria per 20 epithelial cells was counted in 10 microscopic fields. The mean and standard error of adherent bacteria per 20 epithelial cells was calculated. Each adhesion assay was performed in duplicate.

Similar results were observed using non-viable (UCC118 cells heat-killed at 80° C./10 mins).

Scanning Electron Microscopy

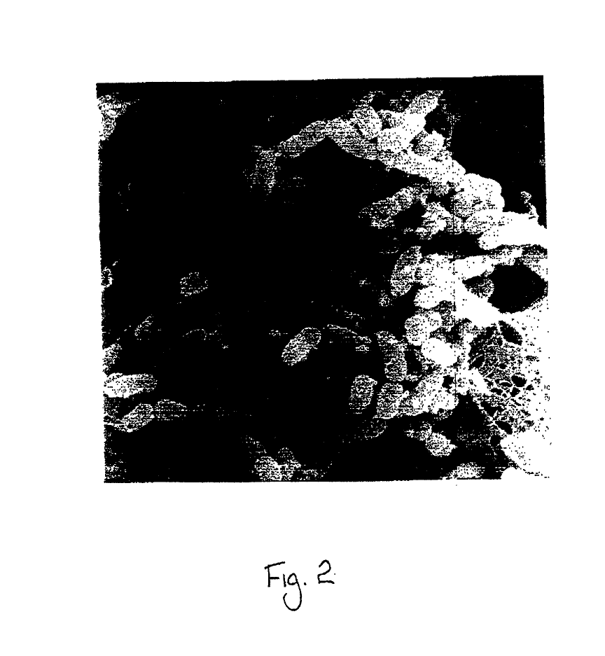

HT-29 cells were grown up on glass discs. After the bacterial adhesion assay, cells were fixed with 2.5% gluteraldehyde in 0.1M phosphate buffer (pH 7.4) for 1 h at room temperature. After two washes with phosphate buffer, cells were postfixed for 30 min with 2% OsO₄ in the same buffer, washed twice with phosphate buffer, and dehydrated in a graded series (30, 50, 70, 80, 90, and 100%) of ethanol. Cells were dried in a critical-point dryer (Balzers CPD030) and coated with gold. The specimens were examined with a Joel JSM 25S scanning electron microscope (FIG. 2).

Characterisation of Adhesion Factor(s)

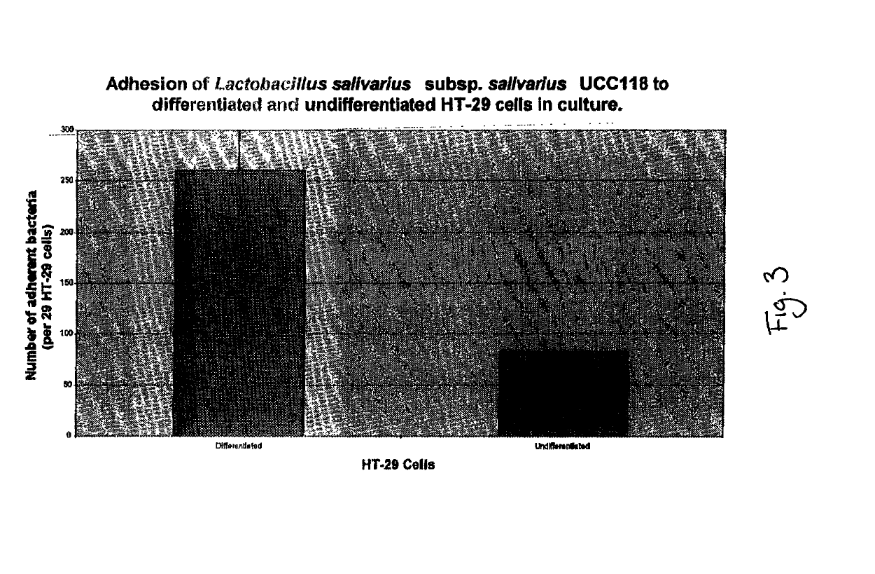

To compare adhesion of Lactobacillus UCC118 to differentiated and undifferentiated HT-29 cells in culture, the monolayers were grown up for 3 days on the glass coverslips before the adhesion assay was performed (as described above). UCC118 was observed to adhere at significantly greater levels to the more physiologically relevant differentiated cells (FIG. 3).

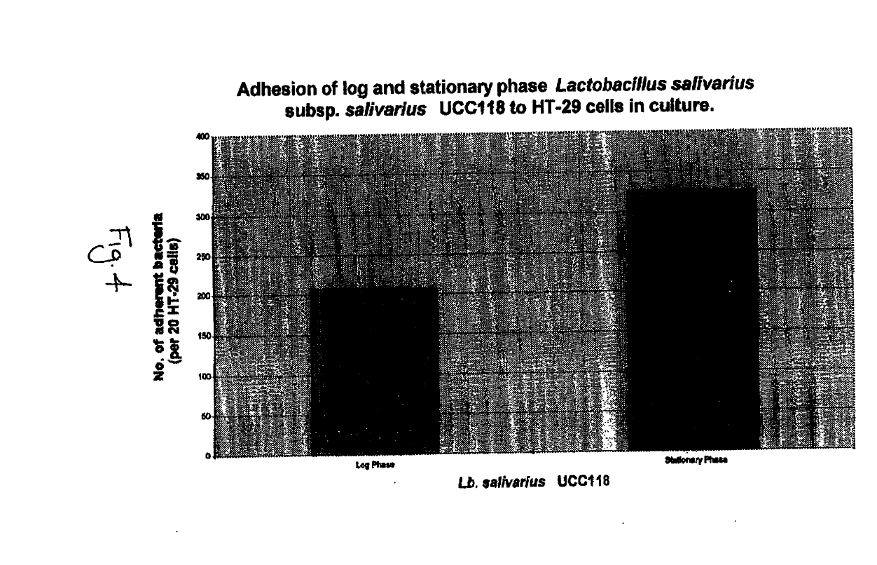

When determining that UCC118 demonstrates greater in vitro adherence in stationary phase of growth rather than log phase (FIG. 4), 6 h cultures were used for the assay.

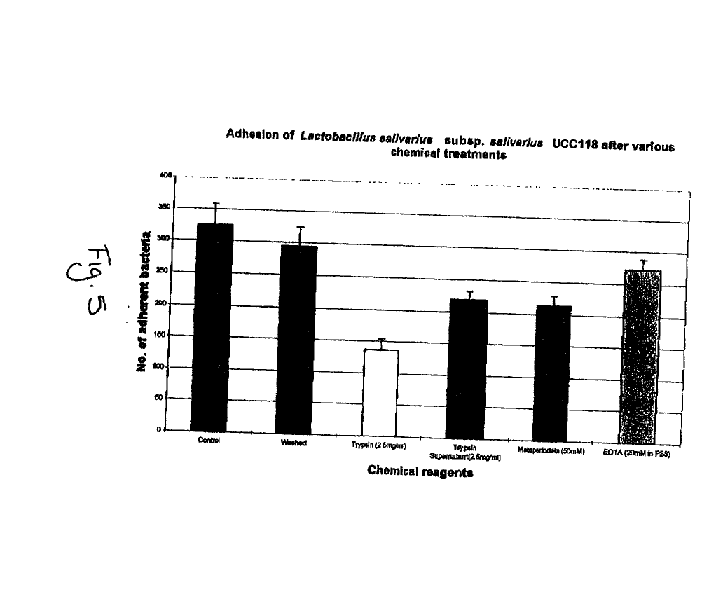

To define the components involved in the adhesion process, the Lb. salivarius UCC118 strain and its spent supernatant were subjected to various chemical treatments (FIG. 5). All chemicals and enzymes were obtained from Sigma Chemical Co. (St. Louis, Md.). Bacterial cells and spent culture supernatant were separated by centrifugation. The cells were washed twice in quarter strength Ringer's solution and re-suspended in MRS broth. In another experiment, the bacterial cells were treated with trypsin (2.5 mg/ml for 60 min at 37° C.), washed and re-suspended in MRS broth. The spent culture supernatant was also treated with trypsin under identical conditions. The trypsin was inactivated by the addition of heat-inactivated (60° C., 30 min) FOETAL CS before the adhesion assay. Bacterial cells were also pre-incubated with metaperiodate (50 mM, 30 min), washed and re-suspended as before. Finally, the HT-29 monolayer was washed five times with 20 mM ethylene diamine tetraacetic acid (EDTA) in PBS after incubation with the bacterial cells.

Washing Lb. salivarius UCC118 in Ringer's solution before the adhesion assay had no effect on the strain's adherence abilities to HT-29 cells in culture. Pre-incubating this strain with trypsin resulted in a highly significant loss of adhesion, while treatment of the spent culture supernatant in the same way did not have such an effect, indicating that a proteinaceous factor is involved in mediating adhesion of Lb. salivarius UCC118 to epithelial cells. Furthermore, this trait appears to be bacterial cell surface-associated, and factors secreted into the surrounding growth medium are not essential. Metaperiodate treatment, to determine the involvement of carbohydrate structures in the adhesion process, resulted in a slight decrease in adherence of the strain, while washing with EDTA after adhesion did not effect Lb. salivarius UCC118 binding significantly, suggesting that calcium is not necessary for adherence of Lb. salivarius UCC118 to occur (FIG. 5).

### EXAMPLE 3

Isolation of Cell-Wall Associated Proteins

The cell wall associated proteins of control and trypsin-treated Lb. salivarius UGC118 cells were extracted using a specifically developed procedure which did not alter the cell membranes.

Bacterial cells (50 ml) were grown up overnight in MRS broth. Control and trypsin-treated cells were washed three times in quarter strength ringers. The cells were then re-suspended in 2 ml TEL reagent (100 mM Tris-HCl pH8, 5 mM EDTA and 0.5-1.0 % lysozyme) and incubated for 3 h at 37° C. The supernatant was collected after centrifugation.

PAGE was performed in the presence of 10% SDS, on the supernatants obtained using the method previously described by Laemmli (1970)(36). Briefly, the supernatants were mixed with a sample buffer (1:4 dilution) containing 5% β-mercaptoethanol, and heated above 90° C. for 10 min. The samples were loaded in the wells of a 5% stacking gel and run at 20 mA until they had passed into a 10% running gel, whereupon the current was increased to 40 mA. Running buffer: Tris 30.26 g, glycine 144.1 g, SDS 10 g and up to 1 L with d.H₂O. Once the molecular weight markers reached the end of the gel, it was stained overnight with comassie blue (0.3 g comassie blue; 100 ml acetate; 100 ml methanol and up to 1 L d.H₂O). Destaining over several hours with regular changes of the destain (300 ml methanol; 80 ml acetate; 620 ml d. H₂O). Protein standards and their molecular weights included the following: α₂-macroglobulin (195 kDa); β-galactosidase (112 kDa); fructose-6-phosphate kinase (84 kDa); pyruvate kinase (63 kDa); fumerase (52.5 kDa); lactic dehydrogenase (35 kDa) and triosephosphatase isomerase.

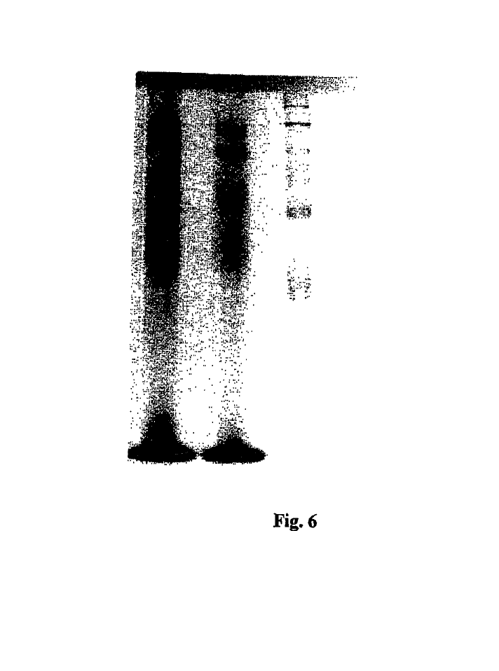

The proteolytic treatment of the bacterial cells resulted in the degradation of two cell wall associated proteins of approximately 195 kDa and 83 kDa (FIG. 6).

### EXAMPLE 4

Assessment of the Influence of FPLC-derived UCC118 Cell-wall Associated Proteins on Adhesion by UCC118 Cells

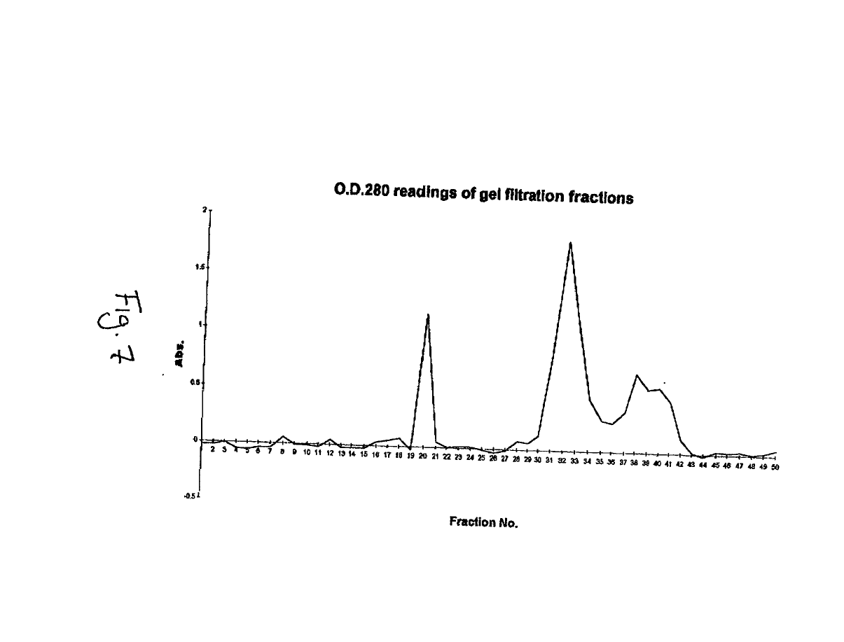

UCC118 was grown overnight in MRS liquid at 37° C. Cultures were centrifuged at 3000 g and supernatants were filter-sterilised and concentrated (×20) by filtration through “Centricon” spin columns with a 10 kDa cut-off (Amicon, USA). Concentrates were dialysed against 50 mM Tris-HCl (pH 7.5) for 4 h, and proteins were separated on a DEAE-Sephacel anion exchange chromatography column (1.6×20 cm; Pharmacia Biotech AB, S-75182 Uppsala, Sweden) equilibrated with the same buffer. Bound proteins were eluted with a gradient between 0 and 500 mM NaCl in the same Tris-HCl buffer. Ten ml fractions were collected using the Fast Protein Liquid Chromatography (FPLC) “Gradifrac” system (Pharmacia). Each fraction was assessed for protein content by measuring the optical density (OD) at 280 nm (FIG. 7).

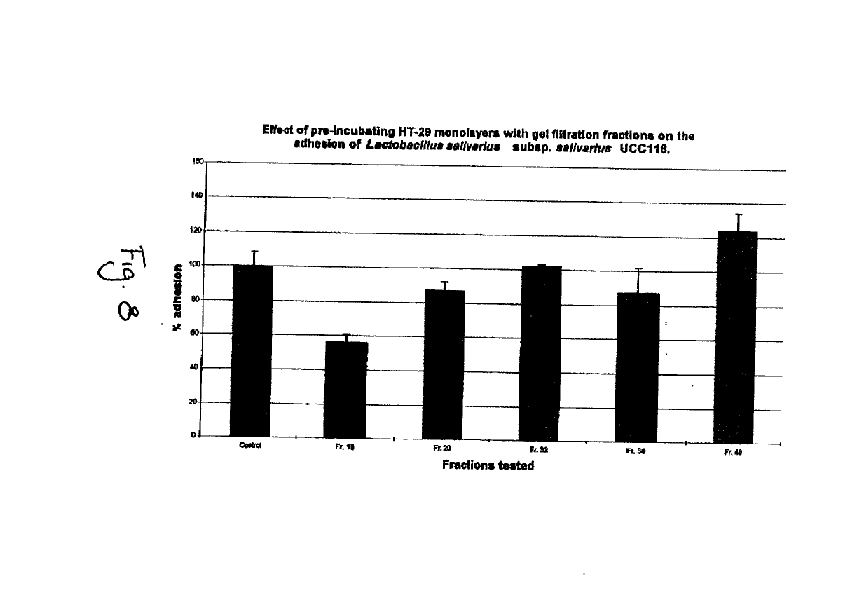

Using the methodologies described above, only FPLC Fraction 18 caused significant reduction in adhesion of strain UCC118 when added prior to the introduction of the bacterial cells (FIG. 8). This is most probably due to binding of available sites on the monolayer used for bacterial attachment.

SDS-PAGE analysis of the FPLC-derived UCC118 cell-wall associated proteins.

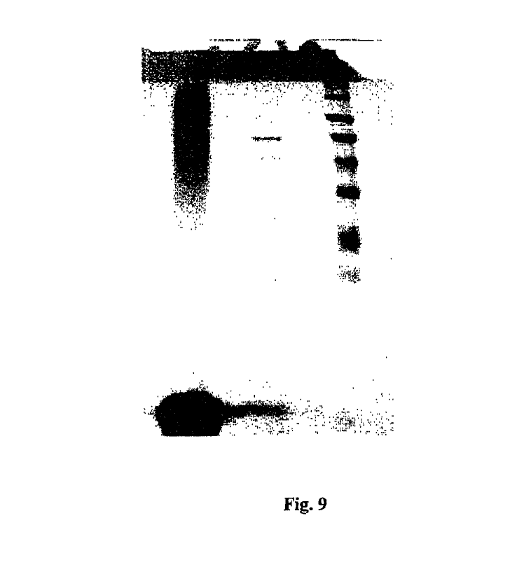

PAGE of FPLC Fraction 18 was performed in the presence of 10% SDS, using the method described above (FIG. 9). It was found that the size of Fraction 18 (approximately 83 kDa) identifies the protein band as the smaller of the proteinaceous components visible in FIG. 6.

N-terminal amino acid sequence analysis.

N-terminal amino acid sequence analysis determined that the UCC118 cell-wall associated protein contains the sequence: WAFRTLILVKADQVSLAKNG.

### EXAMPLE 5

Competitive Exclusion of Potential Pathogen Adherence in vitro by Lactobacillus salivarius UCC118

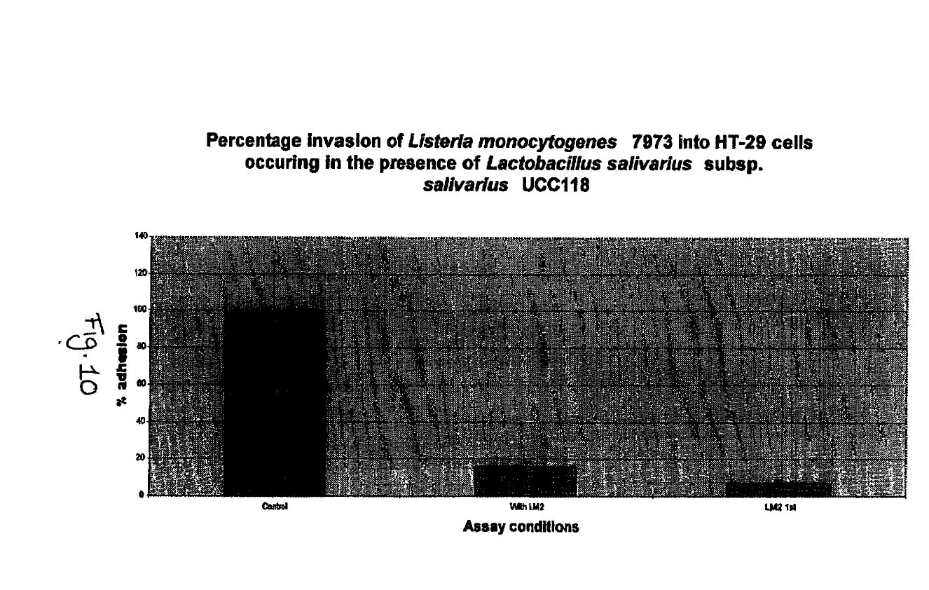

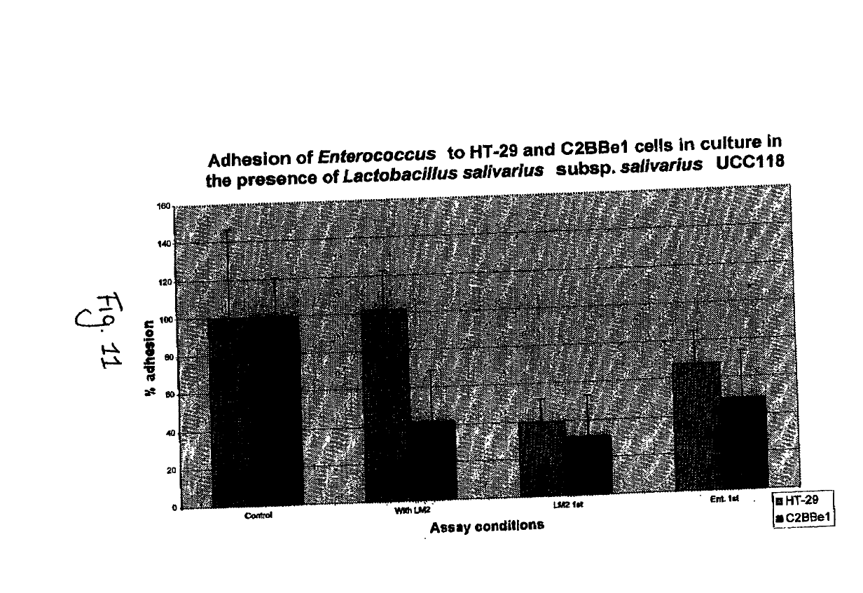

Using the adherence methodologies described in Example 2, it was observed that UCC118 is capable of significantly inhibiting the adherence, at least in vitro, of potentially-pathogenic microbes such as listeria (FIG. 10) and enterococci (FIG. 11). These results have implications for the prophylactic use of probiotic bacteria (or their components/products) in protecting against foodborne disease.

### EXAMPLE 6

Assessment of Intestinal Adhesion by the Lactobacillus Strain

12 Finnish adult ulcerative colitis patients were recruited to assess the ability of Lact. salivarius UCC118^{rif} to adhere to human intestinal mucosa as the probiotic bacterial strain transit through the human gastrointestinal tract. The patients consumed a fermented milk product (120 g) containing viable Lact. salivarius UCC118^{rif} (10¹⁰ cfu/day) for 12 days. The fermented milk product also contained Streptococcus thermophilus.

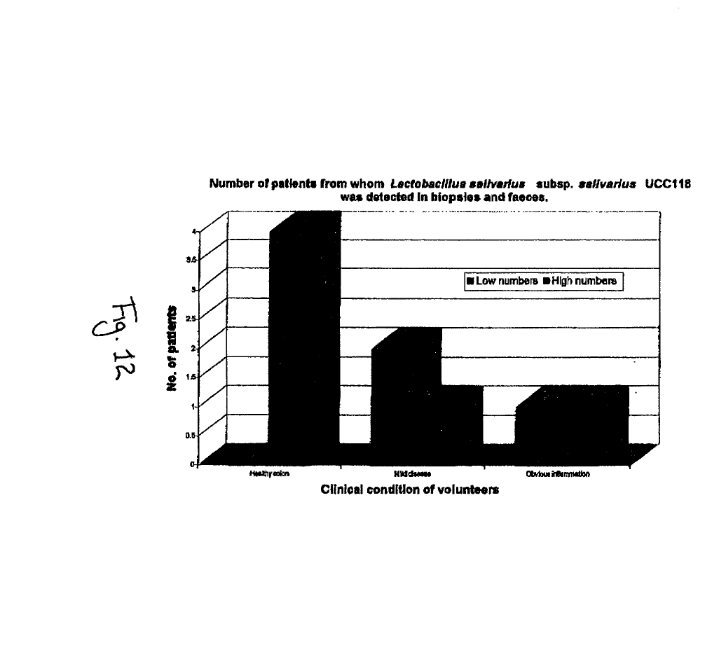

Microbial analysis was performed on endoscopy-derived biopsy samples using MRS medium supplemented with rifampicin (50 μg/ml). Plates were incubated anaerobically in gas pak jars (BBL) with CO₂ generating kits (Anaerocult A;Merck) for 2-5 days at 37° C. No colonies were observed on the antibiotic-containing medium when biopsies were assessed prior to probiotic consumption. However, it was determined that the probiotic bacteria adhered to different anatomical regions of the large bowel and, significantly, to both inflamed and non-inflamed mucosa of the GIT (FIG. 12).

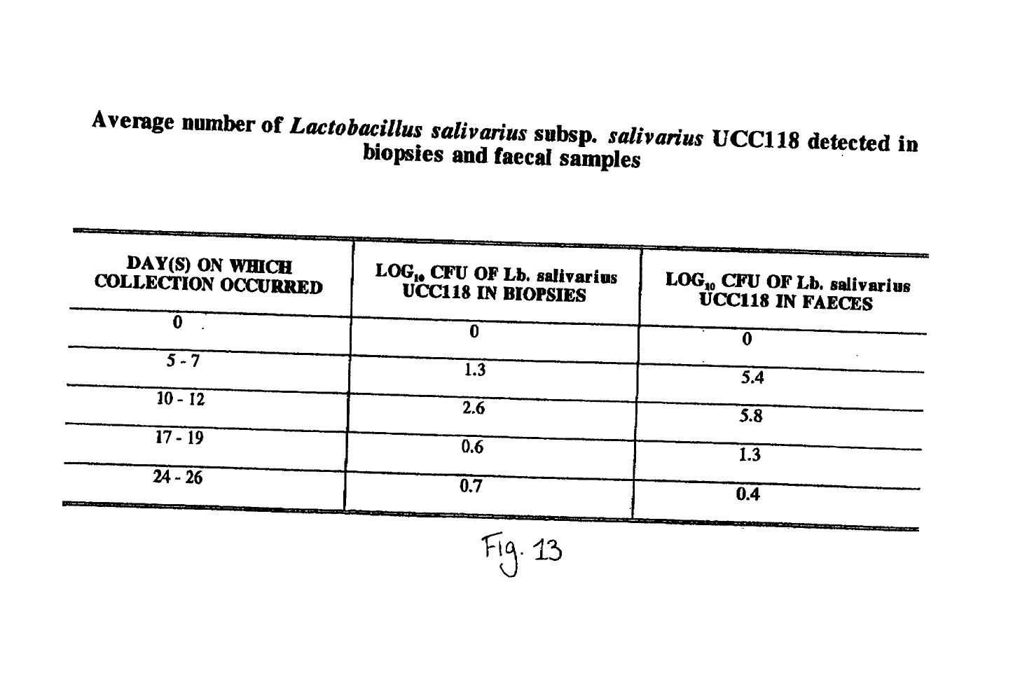

Lb. salivarius UCC118 was found to represent approximately 1-2% of total recoverable lactobacilli from biopsies and faecal samples (FIG. 13) and was capable of persisting on intestinal material for up to 26 days (FIG. 13).

### EXAMPLE 7

Improvement of Epithelial Integrity Following Lactobacillus Adhesion

Lactobacillus UCC118 was added to HT-29 monolayers and allowed to adhere for 4 hours. At this time, monolayers were removed and washed. Following cell lysis, poly (A)⁺ RNA was isolated using magnetic beads. Following quantitation by spectrophotometry, cDNA was generated using specific primers incorporating P³³. Radiolabelled cDNA was purified using spin columns and hybridised to the cellular-interactions array overnight rotating at 65° C. Following a washing procedure, the arrays were exposed to a phosphor screen (Biomax) for 24 hours. The intensity of each gene was calculated from the phosphoimage by comparison to the housekeeping gene β2-microglobulin. Relative mRNA levels for cells stimulated with UCC118 compared to cells that remained non-stimulated were quantified using these arrays. The results are shown in table 1 below.

TABLE 1

| | Fold Increase | Fold Decrease |
|---|---|---|
| Intergrin-α 4 precursor | 360 | |
| Wnt-13 | 150 | |
| Semaphorin CD100 | 110 | |
| Tenascin precursor | 110 | |
| Semaphorin III | 110 | |
| Rac | | 179 |
| TNFα | | 4 |

Interaction between gastrointestinal epithelial cells and probiotic bacteria directly affects intestinal integrity. In this in vitro model, adhesion of this probiotic strain significantly enhanced the expression of cadherins, semaphorins, wnt-13, tenascin and integrins. Cadherins are the prime mediators of epithelial cell-cell adhesion. This has been shown in a murine N-cadherin transgenic model as these mice develop spontaneous colitis (38). Semaphorins play key roles in the control of cellular interactions, while wnt-13 is a developmental protein affecting development of discrete regions of tissue. Tenascin regulates epithelial differentiation and integrins play multiple roles in cell differentiation and cell-cell interactions. Thus UCC118 adhesion results in the upregulation of these epithelial genes improving gut barrier function and gastrointestinal homeostasis.

Following adhesion of UCC118 to HT-29 cells, ras-related C3 botulinum toxin substrate 1 (Rac) and TNFα gene levels were significantly reduced. Rac is a GTPase involved in the invasive behaviour of tumour cells (39). TNFα is a proinflammatory cytokine essential to inflammatory responses. Thus reduction in the levels of these genes would reduce tumour invasiveness and inflammatory responses within the gut.

The invention is not limited to the embodiments hereinbefore described which may be varied in detail.

### REFERENCES

1. Salminen S, Deighton M A, Benno Y & Gorbach S L (1998a) Lactic acid bacteria in health and disease. In: Salminen S., Von Wright, A (Ed) Lactic acid bacteria: Microbiology and functional aspects 2nd Edition (pp 211-54)Marcel Dekker Inc., New York.

2. Salminen S, Bouley C, Boutron-Ruault M-C, Cummings J H, Franck A, Gibson G R, Isolauri E, Moreau M-C, Roberfroid M & Rowland I (1998b) Functional food science and gastrointestinal physiology and function. Brit. J. Nutr. 80(Suppl 1): s147-s171.

3. Fuller R (1989) A review: probiotics in man and animals. J. Appl. Bacteriol. 66: 365-78.

4. Guarner F & Schaafsma G J (1998) Probiotics. Int. J. Food Microbiol. 39: 237-38.

5. Barrow P A, Brooker B E, Fuller R & Newport M J (1980) The attachment of bacteria to the gastric epithelium of the pig and its importance in the microecology of the intestine. J. Appl. Bacteriol. 48: 147-154.

6. Conway P L & Kjelleberg (1989) Protein-mediated adhesion of the Lactobacillus fermentum strain 737 to mouse stomach squamous epithelium. J. Gen. Microbiol. 135: 1175-1186.

7. Hartley C L, Robbins C M & Richmond M H (1978) Quantitative assessment of bacterial adhesion to eukaryotic cells of human origin. J. Appl. Bacteriol. 45: 91-97.

8. Reid G, Servin A L, Bruce A W & Busscher H J (1993) Adhesion of three Lactobacillus strains to human urinary and intestinal epithelial cells. Microbios. 75: 57-65.

9. Takahashi N, Saito T, Ohwada S, Ota H, Hashiba H & Itoh T (1996) A new screening method for the selection of Lactobacillus acidophilus group lactic acid bacteria with high adhesion to human colonic mucosa. Biosci. Biotech. Biochem. 60: 1434-1438.

10. Jacobsen C N, Rosenfeldt Nielsen V, Hayford A E, Moller P L, Michaelsen K F, Paerregaard A, Sandstrom B, Tvede M & Jakobsen M (1999) Screening of probiotic activities of forty-seven strains of Lactobacillus spp. by in vitro techniques and evaluation of the colonization ability of five selected strains in humans. Appl.Environ. Microbiol. 65: 4949-4956.

11. Kirjavainen P V, Ouwehand A C, Isolauri E & Salminen S J (1998). The ability of probiotic bacteria to bind to human intestinal mucus. FEMS Microbiol. Letts. 167: 185-189.

12. Cunliffe D, smart C A, Alexander C & Vulfson E N (1999) Bacterial adhesion at synthetic surfaces. Appl. Environ. Microbiol. 65: 4995-5002

13. Brassart D, Schiffrin E, Rochat F, Offord E A, Macé C & Neeser J-R (1998) The future of functional foods: Scientific basis and future requirements. Lebensmittel Technol. 7-8: 258-66.

14. Kerneis S, Bilge S S, Fourel V, Chauviere G, Coconnier M-H & Dervin A L (1991) Use of purified F1845 fimbrial adhesin to study localization and expression of receptors for diffusely adhering Escherichia coli during enterocytic differentiation of human colon carcinoma cell lines HT-29 and Caco-2 in culture. Infect.Immun. 59: 4013-4018.

15. Eveillard M, Fourel V, Barc M-C, Kerneis S, Coconnier M-H, Karjalainen T, Bourlioux P & Servin A L (1993) Identification and characterization of adhesive factors of Clostridium difficile involved in adhesion to human colonic enterocyte-like Caco-2 and mucus-secreting HT29 cells in culture. Mol. Microbiol. 7: 371-381.

16. Elo S, Saxelin M & Salminen S (1991) Attachment of Lactobacillus casei strain GG to human colon carcinoma cell line Caco-2: comparison with other dairy strains. Letts. Appl. Microbiol. 13: 15-156.

17. Chauviere G, Coconnier M-H, Kerneis S, Fourniat J & Servin A L (1992) Adhesion of human Lactobacillus acidophilus strain LB to human enterocyte-like Caco-2 cells. J. Gen. Microbiol. 138: 1689-1696.

18. Coconnier M-H, Klaenhammer T R, Kerneis S, Bernet M-F & Servin A L (1992) Protein-mediated adhesion of Lactobacillus acidophilus BG2FO4 on human enterocyte and mucus-secreting cell lines in culture. Appl. Environ. Microbiol. 58: 2034-2039

19. Greene J D & Klaenhammer T R (1994) Factors involved in adherence of lactobacilli to human Caco-2 cells. Appl. Environ. Microbiol. 60: 4487-4494.

20. Sarem F, Sarem-Damerdji L O & Nicolas J P (1996) Comparison of the adherence of three Lactobacillus strains to Caco-2 and Int-407 human intestinal cell lines. Letts. Appl. Microbiol. 22: 439-442.

21. Lehto E M & Salminen S (1997) Adhesion of two Lactobacillus strains, one Lactococcus and one Propionibacterium strain to cultured human intestinal Caco-2 cell line. Bioscience Microflora 16: 13-17.

22. Tuomola E M & Salminen S (1998) Adhesion of some probiotic and dairy Lactobacillus strains to Caco-2 cell cultures. Int. J. Food Microbiol. 41: 45-51

23. Alander M, Korpela R, Saxelin M, Vilpponen-Salmela T, Mattila-Sandholm T & von Wright A (1997) Recovery of Lactobacillus rhamnosus GG from human colonic biopsies. Letts. Appl. Microbiol. 24: 361-364.

24. Alander M, Satokai R, Korpela R, Saxelin M, Vilpponen-Salmela T, Mattila-Sandholm T & von Wright A (1999) Persistence of colonization of human colonic mucosa by a probiotic strain, Lactobacillus rhamnosus GG, after oral consumption. Appl. Environ. Microbiol. 65: 351-354.

25. Chauviere G, Coconnier M-H, Kerneis S, Darfeuille-Michaud A, Joly B & Servin A L (1992) Competitive exclusion of diarrheagenic Escherichia coli (ETEC) from human enterocyte-like Caco-2cells by heat-killed Lactobacillus. FEMS Microbiol. Letts. 91: 213-218.

26. Bernet M F, Brassart D, Neeser J R & Servin A L (1994) Lactobacillus acidophilus LA1 binds to cultured human intestinal cell lines and inhibits cell attachment and cell invasion by enterovirulent bacteria. Gut 35: 483-489.

27. Reid G, Tieszer C & Lam D (1995) Influence of lactobacilli on the adhesion of Staphylococcus aureus and Candida albicans to fibers and epithelial cells. J. Indust. Microbiol. 15: 248-253.

28. Tuomola E M, Ouwehand A C & Salminen S (1999) The effect of probiotic bacteria on the adhesion of pathogens to human intestinal mucus. FEMS Immunol.Med. Microbiol. 26: 137-142.

29. Reniero R, Morelli L, Callegari M L, Sommi P & Bottazzi V (1990) Surface proteins in enteric lactobacilli. Ann. Microbiol. 40: 83-91.

30. Klein G, Pack A, Bonaparte C & Reuter G (1998) Taxonomy and physiology of probiotic lactic acid bacteria. Int. J. Food Microbiol. 41: 103-125.

31. Wiley Navarre W & Schneewind O (1999) Surface proteins of Gram-positive bacteria and mechanisms of their targeting to the cell wall envelope. Microbiol. Mol. Biol. Rev. 63: 174-229].

32. Granato D, Perotti F, Masserey I, Rouvet M, Golliard M, Servin A & Brassart D (1999) Cell surface-associated lipotechoic acid acts as an adhesion factor for attachment of Lactobacillus johnsonii La1 to human enterocyte-like Caco-2 cells. Appl. Environ. Microbiol. 65: 1071-1077.

33. Chauviere G, Coconnier M-H, Kerneis S, Fourniat J & Servin Al (1992) Adhesion of human Lactobacillus acidophilus strain LB to human enterocyte-like Caco-2 cells. J. Gen. Microbiol. 138: 1689-1696.

34. Henriksson A, Szewzyk R & Conway P L (1991) Characteristics of the adhesive determinants of Lactobacillus fermentum 104. Appl. Environ. Microbiol. 57: 499-502.

35. Neeser J R, Chambaz A, Golliard M, Link-Amster H, Fryder V & Kolodziejczyk E (1989) Adhesion of CFA/II-positive enterotoxigenic HT-29 cells: a basis for host pathogen interactions in the gut. Infect Immun. 57: 3727-34.

36. Laemmli, U. K. (1970). Nature 227, 680-685.

37. Huis in't Veld J, Shortt C. Selection criteria for probiotic micro-organisms. In: Leeds, A. R., Rowland, I. R. eds. Gut Flora and Health-Past, Present and Future. London: The Royal Society of Medicine Press Ltd., 1996:19-26.

38. Hermiston M. L. & Gordon J. I. Inflammatory bowel disease and adenomas in mice expressing a dominant negative N-cadherin. Science 1995; 270: 1203-1207.

39. Imamura F., Mukai M., Ayaki M., Takemura K., Horai T., Shinkai K., Nakamura H. & Akedo H. Involvement of small GTPases Rho and Rac in the invasion of rat ascites hepatoma cells. Clin. Exp. Metastasis 1999; 17(2): 141-148.

1. An adherence factor comprising a cell wall associated adhesin derived from a Lactobacillus or a derivative, fragment precursor or mutant of the adhesin, the adherence factor mediating adherence to epithelial cells and modulating epithelial gene expression to improve gut barrier function.

2. A factor as claimed in claim 1 wherein expression of a cadherin is upregulated.

3. A factor as claimed in claim 1 wherein expression of any one or more of a cadherin, a semaphorin, wnt-13, tenascin or an integrin is upregulated.

4. A factor as claimed in any of claims 1 to 3 wherein expression of any one or more of ras-related C3 botulinum toxin substrate 1 (Rac) or TNFα is downregulated.

5. A factor as claimed in any preceding claim wherein the Lactobacillus is isolated from resected and washed human gastrointestinal tract.

6. A factor as claimed in any preceding claim wherein the Lactobacillus is Lactobacillus salivarius.

7. A factor as claimed in any preceding claim wherein the Lactobacillus is Lactobacillus salivarius subspecies Salivarius.

8. A factor as claimed in any preceding claim wherein the Lactobacillus is Lactobacillus salivarius subspecies Salivarius UCC118 or a mutant or variant thereof.

9. A factor as claimed in any preceding claim which is proteinaceous.

10. A factor as claimed in any preceding claim having a molecular weight of approximately 83 kDa.

11. A factor as claimed in any preceding claim containing at least portion of the N-terminal amino acid sequence listed in SEQ. ID. No. 1.

12. A factor as claimed in any preceding claim wherein the Lactobacillus is in the form of viable cells.

13. A factor as claimed in any of claims 1 to 11 wherein the Lactobacillus is in the form of non-viable cells.

14. A formulation which comprises a factor as claimed in any of claims 1 to 13.

15. A formulation as claimed in claim 14 which comprises a probiotic material.

16. A formulation as claimed in claim 14 or 15 which comprises a prebiotic material.

17. A formulation as claimed in any of claims 14 to 16 which comprises a strain of Streptococcus thermophilus.

18. A formulation as claimed in any of claims 14 to 17 which comprises an ingestable carrier.

19. A formulation as claimed in claim 18 wherein the ingestable carrier is a pharmaceutically acceptable carrier such as a capsule, tablet or powder.

20. A formulation as claimed in claim 19 wherein the ingestable carrier is a food product such as acidified milk, yoghurt, frozen yoghurt, milk powder, milk concentrate, cheese spreads, dressings or beverages.

21. A formulation as claimed in any of claims 14 to 20 comprising a protein and/or peptide, in particular proteins and/or peptides that are rich in glutamine/glutamate, a lipid, a carbohydrate, a vitamin, mineral and/or trace element.

22. A formulation as claimed in claims 14 to 21 which comprises an adjuvant.

23. A formulation as claimed in claims 14 to 22 which comprises a bacterial component.

24. A formulation as claimed in claims 14 to 23 which comprises a drug entity.

25. A formulation as claimed in claims 14 to 24 which comprises a biological compound.

26. A formulation as claimed in claims 14 to 25 in an orally ingestable form.

27. A factor as claimed in any of claims 1 to 13 or a formulation as claimed in any of claims 14 to 26 for use in foodstuffs.

28. A factor as claimed in any of claims 1 to 13 or a formulation as claimed in any of claims 14 to 26 for use as a medicament.

29. A factor as claimed in any of claims 1 to 13 or a formulation as claimed in any of claims 14 to 26 for use in the prophylaxis and/or treatment of undesirable inflammatory activity.

30. Use of Lactobacillus bacteria isolated from resected and washed human gastrointestinal tract or its cell wall associated adhesin or derivative, fragment, precursor, mutant or recombinant products thereof for improving gut barrier function and or competitively excluding potential pathogens from binding to and or invading epithelial cells.

31. Use of Lactobacillus bacteria isolated from resected and washed human gastrointestinal tract or its cell wall associated adhesin or derivative, fragment, precursor, mutant or recombinant products thereof for mediating adherence of microorganisms to epithelial cells.

32. Use of Lactobacillus as claimed in claim 30 or 31 wherein the Lactobacillus is Lactobacillus salivarius.

33. Use of Lactobacillus as claimed in any of claims 30 to 32 wherein the Lactobacillus is Lactobacillus salivarius subsp. Salivarius strain.

34. Use of Lactobacillus as claimed in any of claims 30 to 33 wherein the Lactobacillus is Lactobacillus salivarius subsp. Salivarius strain UCC118.

35. Lactobacillus salivarius subsp. Salivarius strain or its adhesin component or recombinant products bearing all or part of the adhesin amino acid sequence SEQ. ID. No. 1 for use in engineering hyper-adhesive variants of microorganisms.

36. A vaccine comprising an adherence factor as claimed in any of claims 1 to 13 or a formulation as claimed in any of claims 14 to 26.

37. Use of an adherence factor as claimed in any of claims 1 to 13 for the preparation of a medicament for use in generating an immune response.

38. Use of an adherence factor as claimed in any of claims 1 to 13 for engineering hyperadhesive mutants.

39. Use of an adherence factor as claimed in any of claims 1 to 13 for the preparation of a medicament for use in regulating cell cycle and/or invasive behaviour of tumour cells.

40. A delivery system for delivery of bourne factors to intestinal tissue comprising a factor as claimed in any of claims 1 to 13.

41. An adhesin component derived Lactobacillus salivarius subsp. Salivarius strain UCC118 or recombinant products bearing all or part of the adhesin amino acid sequence SEQ. ID. No. 1 for use in generating an immune response in inflamed and/or non-inflamed intestinal tissue.

42. Lactobacillus salivarius subsp. Salivarius strain UCC118 or its adhesin component or recombinant products bearing all or part of the adhesin amino acid sequence SEQ. ID. No. 1 for use as a vaccine.

43. Lactobacillus salivarius subsp. Salivarius strain UCC118 or its adhesin component or recombinant products bearing all or part of the adhesin amino acid sequence SEQ. ID. No. 1 for use in the delivery of borne factors to inflamed and/or non-inflamed intestinal tissue and persistence at the sites of adherence to allow slow-release of the borne factors.

44. Lactobacillus salivarius subsp. Salivarius strain UCC118 or its adhesin component or recombinant products bearing all or part of the adhesin amino acid sequence SEQ. ID. No. 1 for use in foods or medicaments.

45. A cell wall associated adhesin having a molecular weight of approximately 83 kDa.

46. A cell wall associated adhesin containing the N-terminal amino acid sequence listed in SEQ. ID. No. 1.


SEQUENCE LISTING (1 sequence)

SEQ ID NO: 1 - protein, 20 aa, Lactobacillus salivarius
WAFRTLILVKADQVSLAKNG